(19) United States
(12) Patent Application Publication
(10) Pub. No.: US 2011/0129424 A1
(43) Pub. Date: Jun. 2, 2011

(54) FLUORINATED POLYMERS AND ASSOCIATED METHODS

(76) Inventors: Cory Berkland, Lawrence, KS (US); Mark Bailey, Mountain View, CA (US)

(21) Appl. No.: 12/883,943

(22) Filed: Sep. 16, 2010

Related U.S. Application Data
(60) Provisional application No. 61/242,907, filed on Sep. 16, 2009.

(51) Int. Cl. A61K 49/00; C08F 220/24; C08F 8/32; C08F 8/40; B82Y 5/00

(52) U.S. Cl. 424/9.6; 526/245; 424/9.1; 525/54.1; 525/54.2; 525/326.2; 428/402; 977/773; 977/788; 977/927

(57) ABSTRACT

Compositions comprising a fluorinated polymer and related methods.

### CROSS-REFERENCE TO RELATED APPLICATIONS

This application claims priority to U.S. Provisional Patent Application Ser. No. 61/242,907, filed Sep. 16, 2009, the entire disclosure of which is incorporated by reference.

### BACKGROUND

Nanomaterials have emerged as valuable tools in biomedical imaging techniques, such as magnetic resonance imaging (MRI), fluorescence, and positron emission tomography (PET). Some have been designed to serve as multimodal imaging agents, combining seemingly disparate techniques such as fluorescence, photoacoustic tomography (PAT), or positron emission tomography (PET) with magnetic resonance imaging (MRI). Particles have also been designed that combine PET with fluorescence imaging and magnetic resonance imaging capabilities. Combining different imaging modalities into a single contrast agent enables one to capture meaningful images at different levels of spatial resolution from the same sample.

Fluorinated nanoparticles have also been studied as potential MRI contrast agents. The rarity of physiologic fluorine can be exploited to generate highly selective, ¹⁹F images that can be superimposed over complimentary ¹H images, thus providing an anatomical context for the fluorinated contrast agent. Research in this area has focused on the synthesis of fluorinated nanoparticles using a variety of different approaches. Studies have validated the premise of ¹⁹F MRI using nanoemulsions that contain a perfluorocarbon phase. These studies have used materials such as perfluoro-15-crown-5-ether or perfluorooctyl bromide, which is emulsified into an aqueous phase using a biocompatible surfactant. Other studies have created novel fluorinated amphiphilic block copolymers that form micelles in aqueous solution. Typically, a living free radical polymerization method is employed to synthesize such amphiphilic block copolymers. This polymer self assembles in water and the resulting suspension is used as the contrast agent.

Although current methods to create fluorinated nanoparticles have demonstrated some success, a potential concern is nanoparticle stability. For example, the stability of some micellar nanoparticles and lipid-based emulsions is dependent on the critical micellar concentration (CMC) of the material. Concentration can easily be controlled in vitro, but the concentration of the particles might vary greatly when introduced into a physiological environment, which could disrupt the micelle or nanoemulsion stability if the local concentration falls below a critical concentration, which could adversely affect in vivo performance.

### SUMMARY

The present disclosure, according to specific example embodiments, generally relates to fluorinated polymers, methods of making fluorinated polymers, and methods of using fluorinated polymers.

The features and advantages of the present disclosure will be apparent to those skilled in the art. While numerous changes may be made by those skilled in the art, such changes are within the spirit of the invention.

### DRAWINGS

A more complete understanding of this disclosure may be acquired by referring to the following description taken in combination with the accompanying figures.

FIG. 1 depicts a schematic representation of an embodiment of nanoparticle synthesis. The middle scheme shows the proposed interaction between fluorinated side chains as the particles are transferred from ethanol to water and goes from transparent to turbid. The photograph inset shows (A) reagent mixture prior to reaction, (B) nanoparticle suspension in ethanol after reaction, and (C) nanoparticle suspension in water after dialysis and centrifugation. (PVP, polyvinylpyrrolidone; EtOH, ethanol; Vazo-52, (E)-2,2′-(diazene-1,2-diyl)bis(2,4-dimethylpentanenitrile).

FIG. 2: Panel (A) shows SEM images of the fluorinated nanoparticles. The “craters” are bubbles in the residual PVP, which has deposited as a thin matrix. The white spheres and ellipsoids are nanoparticles, some of which are trapped in residual PVP. Panel (B) shows dynamic light scattering measurements of fluorinated nanoparticles under different conditions. The top graph shows size as a function of nanoparticle concentration. The bottom graph shows the effect of Tween-20 and sonication on particle size. Under shear and in the presence of the surfactant, particle flocculation is reduced.

FIG. 3: FTIR spectrum of fluorinated nanoparticles. Panel (A) shows the spectrum for PVP, (B) shows the spectrum for particles synthesized in PVP, and (C) shows the spectrum for particles synthesized without PVP. Both panels (B) and (C) show amide I and amide III peaks, suggesting that NVF is incorporated into the particles. Panels (B) and (C) also show carbonyl peaks and ester peaks, suggesting the presence of the fluorinated ester acrylate group.

FIG. 4: Panel (A) shows negative secondary ion mass spectra for nanoparticle samples. Labeled peaks suggest the presence of nitrogen-containing functional groups and fluorinated groups on the surface of the particles. Panel (B) shows the solid state ¹⁹F NMR spectrum of the fluorinated nanoparticles. The locations of the peaks are consistent with the presence of two different fluorine-containing sites in the fluorinated group. The peak at −82.1 ppm originates from CF₃ fluorine, and the one at −122.8 ppm from CF₂ fluorine. The CF₂ peak is surrounded by spinning sidebands.

FIG. 5: Diagram of the reaction scheme. For each batch, one of the fluorinated monomers was reacted with (1,5-N-vinylformamido) ethyl ether and N-vinylformamide to generate nanoparticles. The reaction was carried out in ethanol at 60° C. using Vazo-52 as an initiator and polyvinylpyrrolidone (PVP) as a surfactant. For the analogous polymers, the same reaction scheme was used, but without the addition of the (1,5-N-vinylformamido) ethyl ether crosslinker or the PVP surfactant.

FIG. 6: USANS and SANS spectra of particles prepared with 1H,1H-perfluoro-n-octyl acrylate monomer. Particles were dispersed in D₂O without surfactant at various concentrations. Data were fit using a Schulz sphere analytical model. The model suggests that the particles are between 290 and 414 nm in diameter with a polydispersity of approximately 0.52.

FIG. 7: USANS spectra of particles prepared with 2-(allyl)hexafluoroisopropanol monomer. Particles were dispersed in D2O without surfactant at concentrations of 1 mg/mL and 0.5 mg/mL. Data were fit using a Schulz sphere analytical model. The model suggests that the particles are approximately 282 nm in diameter. The polydispersity was held to 0.5 due to the noisiness of the data.

FIG. 8: TEM Image of particles prepared with 1H,H-perfluoro-n-octyl acrylate monomer (A) and 2-(allyl)hexafluoroisopropanol monomer (B).

FIG. 9: FTIR spectra of fluorinated nanoparticles. (A) Shows particles and (B) shows polymer prepared with 1H,1H-perfluoro-n-octyl acrylate monomer. (C) Shows particles and (D) shows polymer prepared with the 2-(allyl)hexafluoroisopropanol monomer. All solutions were allowed to evaporate prior to analysis of the resultant nanoparticle or polymer film.

FIG. 10: Representative chemical structures of non-hydrolyzed and hydrolyzed particles prepared using the different monomers. (A) Hydrolysis of particles prepared using the 1H,1H-perfluoro-n-octyl acrylate monomer, where amide groups were hydrolyzed to their corresponding amines, and the fluorinated ester was also cleaved. (B) Hydrolysis of particles prepared using the 2-(allyl)hexafluoroisopropanol monomer, where the amide groups were converted to primary and secondary amines, while the fluorinated regions remained intact.

FIG. 11: ¹⁹F-NMR spectra of fluorinated nanoparticles. (A) The spectrum for nanoparticles prepared using the 1H,1H-perfluoro-n-octyl acrylate monomer, which showed a broad peak at −83.35 ppm. (B) The spectrum for nanoparticles prepared using the 2-(allyl)hexafluoroisopropanol monomer, which showed a sharp peak at −76.21 ppm. Insets show the spectra for the entire sweep width.

FIG. 12: Synthesis of fluorinated-fluorescent nanoparticles. For each batch, monomers were dissolved in ethanol containing polyvinylpyrrolidone (PVP) as a surfactant and Vazo-52 initiator. The reaction was carried out at 60° C. for 24 hours.

FIG. 13: Upregulation of ICAM-1 expression in HUVEC cells incubated with TNF-α, as measured by the fluorescence intensity of anti-ICAM-1-FITC antibody. The results suggested an approximately 32×increase in ICAM-1 expression after incubation with TNF-α. Error bars represent the standard deviation, with n=3.

FIG. 14: Normalized fluorescence intensity of LABL-conjugated NPs and non-conjugated NPs in HUVECs. The results suggested a much greater normalized fluorescence intensity for the LABL-conjugated nanoparticles, most likely due to binding facilitated by the LABL peptide. Data are presented by mean±standard deviation. *p<0.05.

FIG. 15: HUVECs after incubation with non-conjugated NPs and LABL-NPs. The results suggested that conjugation with the LABL peptide increases uptake of the particles into the cells.

FIG. 16: Colocalization of untargeted and targeted nanoparticles with lysosomes. Lysosomes were stained with Texas red dextran, and cell nuclei were stained with DAPI (blue). Colocalization of nanoparticles (green) with lysosomes would appear yellow. The results suggested that the nanoparticles were not incorporated within the lysosomes. This could be due to their large size (approximately 300 nm). It is believed that the particles were internalized via endocytosis, but were not incorporated into the lysosomes.

FIG. 17: TOF-SIMS images of A10 rat aorta smooth muscle cells grown on a silicon substrate. Panels (A) and (B) were imaged before sputtering with SF5, and (C) and (D) were imaged after sputtering. (A): Dark purple—amino acids; Light purple—sulfates; Green—phosphates. (B): Yellow—phospholipids; Blue—amino acids. (C): Yellow—amines and phosphates; Green—amines; Red—phosphates; Blue—silicon substrate. (D): Yellow—amino acids and phosphates; Green—cholesterol, amino acids and phosphates; Blue—silicon substrate.

The patent or application file contains at least one drawing executed in color. Copies of this patent or patent application publication with color drawing(s) will be provided upon request and with payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as defined by the appended claims.

### DESCRIPTION

The present disclosure, according to specific example embodiments, generally relates to fluorinated polymers. Such fluorinated polymers may be particles, such as nanoparticles. In some embodiments, fluorinated polymers of the present disclosure may be used as imaging contrast agents, for example, in biomedical imaging techniques, such as magnetic resonance imaging (MRI), fluorescence, positron emission tomography (PET), secondary ion mass spectrometry (SIMS), and others. The fluorinated polymers of the present disclosure may be used for surface modification, for example, adding charge to hydrophobic surfaces. The fluorinated polymers also may be used for enhancing contrast in imaging, for example, MRI, SIMS, and the like.

The present disclosure provides, according to certain embodiments, a composition comprising a fluorinated polymer. In some embodiments, the fluorinated polymer may be prepared from a polymerization reaction comprising at least one hydrophilic monomer and at least one fluorinated monomer, so as to form a fluorinated polymer comprising a hydrophilic monomer component and a fluorinated monomer component.

A variety of fluorinated monomers may be used to form the fluorinated polymer compositions of the present disclosure. In some embodiments, suitable fluorinated monomers may include any polymerizable monomer comprising fluorine. Examples of suitable fluorinated monomers include, but are not limited to, tetrafluoroethylene (TFE), hexafluoropropylene (HFP), chlorotrifluoroethylene (CTFE), trifluoroethylene, hexafluoroisobutylene, perfluoroalkyl ethylenes, fluorovinyl ethers, vinyl fluoride (VF), vinylidene fluoride (VF2), perfluoro-2,2-dimethyl-1,3-dioxole (PDD), perfluoro-2-methylene-4-methyl-1,3-dioxolane (PMD), perfluoro(allyl vinyl ether), perfluoro(butenyl vinyl ether), and 2-(allyl)hexafluoroisopropanol.

Suitable fluorinated monomers may also include non-acrylic fluorinated monomers and acrylic fluorinated monomers. Examples of non-acrylic fluorinated monomers may include, but are not limited to fluorostyrenes such as, 2,3,4,5,6-Pentafluorostyrene, 2-fluorostyrene, 3-fluorostyrene, 4-fluorostyrene, 2,6-difluorostyrene, 2-(trifluoromethyl)styrene, 3-(trifluoromethyl)styrene, and 4-(trifluoromethyl)styrene. Examples of acrylic fluorinated monomers may include, but are not limited to, 2,2,2-trifluoroethyl methacrylate, 1H,1H-perfluoro-n-octyl acrylate, 1H,1H-perfluoro-n-decyl acrylate, 1H,1H-perfluoro-n-octyl methacrylate, 1H,1H-perfluoro-n-decyl methacrylate, hexafluoroisopropyl acrylate, and hexafluoroisopropyl methacrylate.

The hydrophilic monomer may be any hydrophilic monomer capable of polymerizing with the fluorinated monomer. By way of explanation, and not of limitation, it is currently believed that the hydrophilic monomer may enhance the stability of the fluorinated polymer in an aqueous environment. Examples of suitable hydrophilic monomers include, but are not limited to, N-vinylformamide (NVF), acrylates, PEG-acrylates, and the like. In some embodiments the hydrophilic monomer may be degradable or bioresorbable. In some embodiments, the hydrophilic monomer (e.g., acrylamide) may be hydrolyzed to form a carboxylic acid.

In some embodiments, the hydrophilic monomer may be hydrolzyed to yield a charged monomer. For example, a hydrophilic monomer like NVF may be hydrolyzed to form polymers with primary amines to form a cationic polymer.

In some embodiments, a fluorinated polymer of the present disclosure may further comprise a crosslinker. In some embodiments, the crosslinker may comprise a covalent chemical crosslinker. By way of explanation, and not of limitation, such chemical linking may provide increased nanoparticle stability. In some embodiments the crosslinker may be degradable or bioresorbable. In one specific embodiment, a suitable crosslinker for NVF may be (1,5-N-vinylformamido) ethyl ether.

In some embodiments, a fluorinated polymer composition of the present disclosure may be functionalized with a chemical species, for example, to facilitate targeting of the fluorinated polymer to specific cells or tissues. Accordingly, in some embodiments, the fluorinated polymer compositions of the present disclosure may include a targeting moiety. Suitable targeting moieties may be synthetic, partially synthetic or of biological origin. Suitable targeting moieties also may actively or passively target specific cells or tissues, location of specific pathologies, sites of inflammation, sites of specific pathologies, tumors, or non-self components (e.g., viral, bacterial, fungal, protozoan, and the like). Suitable targeting moieties also may include antibodies and RNA fragments specific for a tissue or pathology of interest (e.g., that specifically bind to a tumor cell epitope such as protein epitopes, lipids, sphingolipids, nucleic acid epitopes, carbohydrate groups, or other biological distinct epitopes or antigens).

In certain embodiments, a fluorinated polymer composition may be formed in a single step, free radical polymerization. In other embodiments, a fluorinated polymer composition may be formed in using a sequential path of synthesis.

In some embodiments, a fluorinated polymer composition may be formed into a particle, such as a nanoparticle. In certain embodiments, such fluorinated nanoparticles may have an average particle size of about 1,000 nm or less. In certain embodiments, such fluorinated nanoparticles may have an average particle size of about 500 nm or less. In certain embodiments, the fluorinated nanoparticle may be of a size that allows for long circulation half-life when introduced into a subject. In such embodiments the fluorinated nanoparticle may preferentially extravasate through the discontinuous endothelium of a tumor and into the tumor interstitium, where it could accumulate and provide contrast in that region. This phenomenon is known as the enhanced permeability and retention effect, or EPR effect.

In some embodiments, the fluorinated polymer may be adsorbed onto a hydrophobic surface. Accordingly, in some embodiments, the hydrophilic monomer may improve the hydrophilicity of the hydrophobic surface. The hydrophilic monomer may also be hydrolyzed and may, thereby, impart charge to the hydrophobic surface.

In some embodiments, a fluorinated polymer composition may further comprise a fluorescent compound. Examples of suitable fluorescent compounds may include any compound capable of emitting fluorescence.

To facilitate a better understanding of the present disclosure, the following examples of certain aspects of some embodiments are given. In no way should the following examples be read to limit, or define, the scope of the invention.

### Example 1

Materials: Materials were purchased from Sigma-Aldrich (St. Louis, Mo.) unless otherwise stated. 1H,H-perfluoro-n-octyl acrylate was purchased from ExFluor Research Corporation (Round Rock, Tex.). Vazo-52 was purchased from DuPont (Wilmington, Del.). Prior to nanoparticle synthesis, (1,5-N-vinylformamido) ethyl ether was prepared as previously described. Impurities were precipitated out of N-vinylformamide using absolute ethanol and vacuum filtered prior to use. All other reagents were used as received.

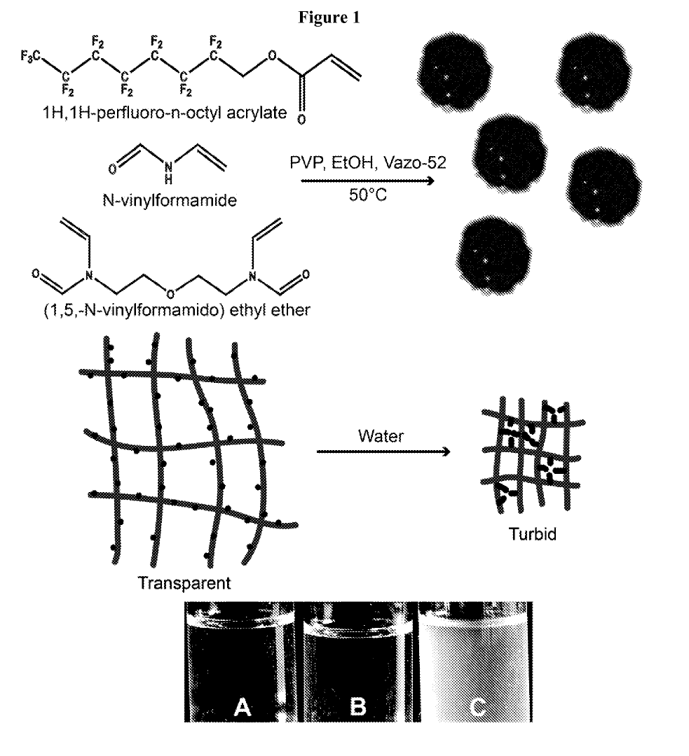

Nanoparticle Synthesis: Nanoparticles were synthesized using a free radical polymerization technique. 10 μL, of 1H,H-perfluoro-n-octyl acrylate, 3.5 μL, of N-vinylformamide, 7 μL, of (1,5-N-vinylformamido) ethyl ether, and 0.005 g of (E)-2,2′-(diazene-1,2-diyl)bis(2,4-dimethylpentanenitrile) (Vazo-52) were added to absolute ethanol containing 0.018 g/mL polyvinylpyrrolidone (MW approximately 360 kDa). The reagent mixture was sparged for 10 min with argon to remove dissolved oxygen, then heated in a silicone oil bath to 50° C. and stirred at approximately 900 rpm. The reaction was carried out under an argon atmosphere for 24 h. The product was then dialyzed against deionized water using a 1 kDa MWCO regenerated cellulose ester dialysis tube for 24 h. The dialysate was changed 5 times to ensure complete solvent exchange. Particle suspensions were then centrifuged twice at 15,000 rpm for 45 min. The pellet was collected each time and resuspended in deionized water. The reaction scheme is shown in FIG. 1.

Characterization of Nanoparticles: The size and zeta potential of the nanoparticles were determined using dynamic light scattering. All measurements were taken 5 times. Measurements are reported as the mean±standard error. SEM experiments were performed using a field emission scanning electron microscope. Samples were prepared by decanting a small volume of nanoparticles suspended in deionized water onto a polished silicon wafer and allowing the water to evaporate under a fume hood. Samples were sputter coated with 5 nm of gold prior to imaging. All samples were analyzed using an acceleration voltage of 10 keV under high vacuum.

Time of Flight Secondary Ion Mass Spectrometry (TOF-SIMS): TOF-SIMS was used to analyze the surface chemistry of the nanoparticles. Samples were prepared by decanting a small volume of nanoparticles suspended in deionized water onto a polished silicon wafer and allowing the water to evaporate under a fume hood. TOF-SIMS experiments were performed on an Ion-TOF IV instrument equipped with both Bi (Biₙ⁺, where n=1 to 7) and SF₅⁺ primary ion beam cluster sources. The analysis source was a pulsed, 25 keV bismuth cluster ion source (Bi₃), which bombarded the surface at an incident angle of 45 degrees to the surface normal. The target current was maintained at approximately 0.3 pA (±10%) pulsed current with a raster size of 200 μm×200 μm for all experiments. Both positive and negative secondary ions were extracted from the sample into a reflectron-type time of flight mass spectrometer. The secondary ions were then detected by a microchannel plate detector with a post-acceleration energy of 10 kV. A low energy electron flood gun was utilized for charge neutralization in the analysis mode. Each spectrum was averaged over a 60 s time period, with a cycle time of 100 μs. These conditions resulted in accumulated Bi₃⁺ ion doses that were well below 10¹³ ions/cm².

FTIR Reflection Spectroscopy: FTIR spectroscopy was used to qualitatively determine the identity of functional groups present within the nanoparticles. All experiments were done on a diamond attenuated total reflectance objective microscope accessory. Reported spectra are the average of 128 scans.

Solid State ¹⁹F-NMR Spectroscopy: The solid-state NMR (ssNMR) spectra were obtained on a 3-channel spectrometer operating at 284.0 MHz for ¹⁹F and 301.9 MHz for ¹H using an ¹H/¹⁹F probe. The sample was packed in a 4 mm zirconia rotor with Torlon™ endcaps and Vespel™ drivetips and rotated at 10,000 kHz. The NMR spectrum was obtained using H—F cross polarization and a sweep width of 100 kHz. A total of 1024 scans were obtained with a dwell time of 10 μs. The chemical shift reference was set at −121.1 ppm using Teflon.™ Interference from the Teflon™ endcaps was not subtracted because it was negligible under these conditions.

Results

Here, the one-step synthesis and characterization of a novel fluorinated, polymeric nanoparticle containing a covalent crosslinker is described.

Fluorinated nanoparticles were synthesized using a single step, free radical polymerization method and then precipitated in water. In this method, N-vinylformamide, (1,5-N-vinylformamido) ethyl ether, and 1H,H-perfluoro-n-octyl acrylate were added to a solution of polyvinylpyrrolidone (PVP) in ethanol (FIG. 1). Vazo-52 was added as an initiator, and the solution was sparged with argon. The reaction was carried out at 50° C. for 24 hours. Particles were prepared without PVP under the same conditions to serve as a control group. These particles were larger than the particles prepared in PVP solution and were not used in further analysis. The product was then dialyzed against deionized water to induce particle precipitation, and then centrifuged and resuspended twice in water (FIG. 1).

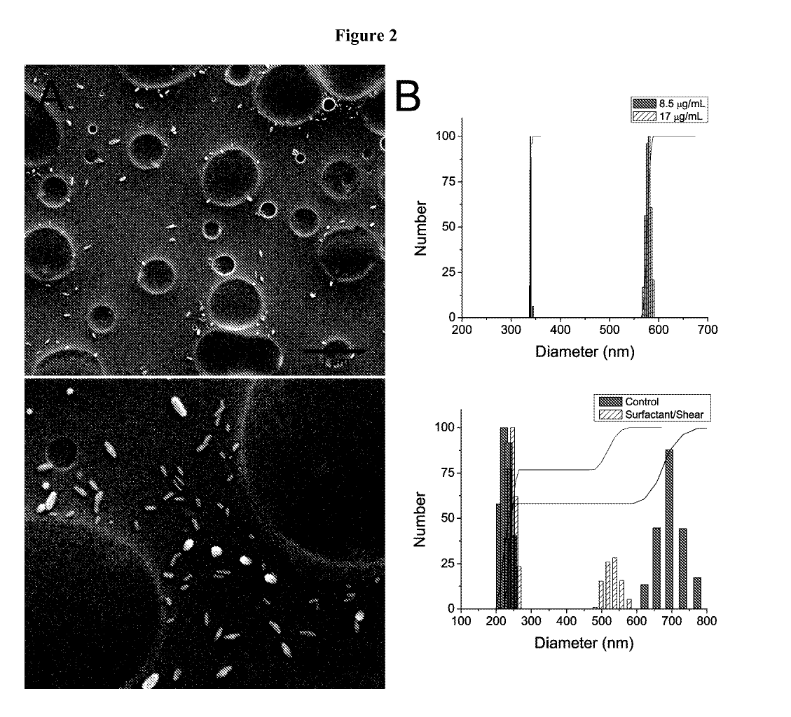

The nanoparticles had a size distribution with maxima at 250 nm and 700 nm according to dynamic light scattering. After adding Tween-20 (final concentration: 5.0×10₋₄ g/mL) and sonicating for 4 h, the distribution maxima shifted to 250 nm and 575 nm (FIG. 2.2). Environmental scanning electron microscopy (ESEM) imaging suggested that the particles were substantially smaller than 500 nm (FIG. 2). The disparity between the ESEM and the DLS data could be partially due to swelling of the particles in aqueous medium, but is most likely due to flocculation occurring in water, which would increase the particle size observed by DLS. DLS measurements taken of the product in ethanol after polymerization were indistinguishable from the background, suggesting that the product was soluble. After precipitation in water and solvent exchange, particles demonstrated excellent colloidal stability, and showed only minor settling when left undisturbed at room temperature for more than five months. This settling was easily reversed by lightly shaking the vial for several seconds.

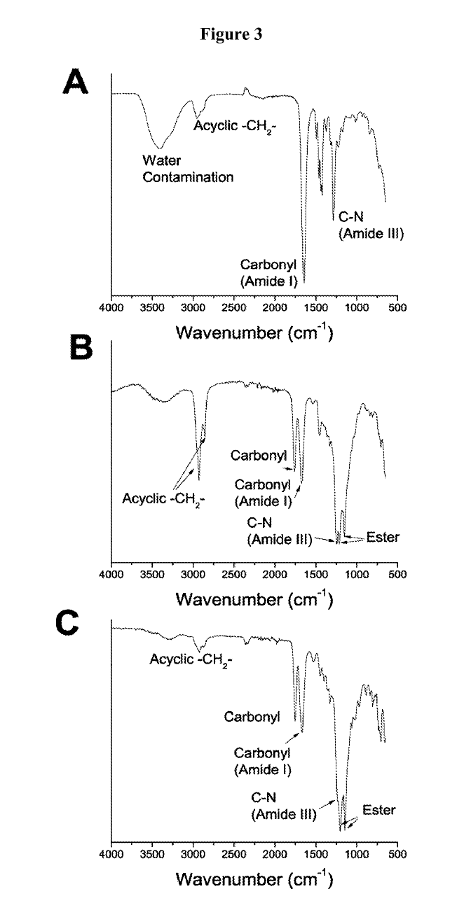

FTIR was used to determine the groups present in the particles (FIG. 3). The spectra for the nanoparticles show bands corresponding to both amide I (1670 cm⁻¹-1650 cm⁻¹) and amide III (1315 cm⁻¹-1250 cm⁻¹) peaks. The spectra also showed a second peak in the carbonyl region (1690 cm⁻¹-1760 cm⁻¹), as well as peaks in the ester region (1080 cm⁻¹-1300 cm⁻¹), which were due to the presence of the fluorinated ester group. These peaks were present in the spectra from particles prepared both with and without PVP surfactant, indicating that they originate from the particles themselves and were not solely an artifact from the γ-lactam groups present in residual PVP.

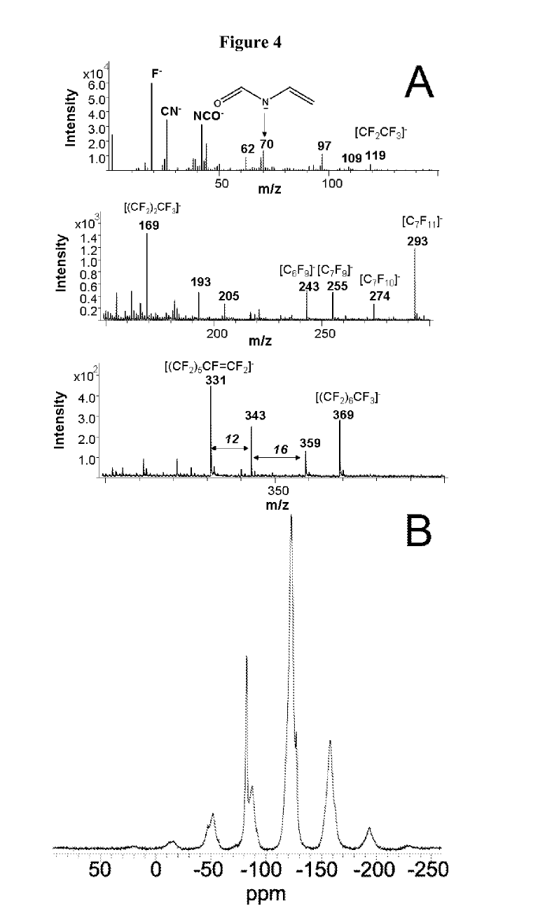

TOF-SIMS experiments also strongly revealed the presence of fluorinated groups on the surface of the particles (FIG. 4), indicating they would be a suitable target for cellular imaging applications. SIMS has a sampling depth of approximately 1 nm in polymeric materials, suggesting that fluorinated residues were present on the surface of the particles. Spectra also indicated the presence of nitrogen-containing groups, which could be from the N-vinylformamide residue, the (1,5-N-vinylformamido) ethyl ether crosslinker residue, the 2,4-dimethylpentanenitrile residue from the initiator, or residual PVP surfactant. Regardless of their source, the nitrogen-containing groups provided the particles with a hydrophilic surface character, which may contribute to their aqueous stability.

The presence of the fluorinated groups on the surface of the particles could help explain the disparity between the particle sizes measured with dynamic light scattering and the sizes suggested from the ESEM experiments. The fluorinated groups are extremely hydrophobic, and it is probable that their presence on the particles' surface would induce flocculation due to hydrophobic Van der Waals interactions. This phenomenon would be in competition with the repulsive effects of the hydrophilic groups on the particles' surface. DLS experiments showed changes in measured particle size as particle concentration was varied, which suggests that flocculation was occurring (FIG. 2). Additionally, sonication and the addition of Tween-20 (final concentration 5.0×10⁻⁴ g/mL) were shown to decrease the effect of flocculation.

Solid-state ¹⁹F-NMR (ssNMR) was used to help further elucidate the structure of the particles (FIG. 4). The locations of the peaks are consistent with the presence of two different fluorine-containing sites within the fluorinated group. The peak at −82.1 ppm originates from CF₃ fluorine and the one at −122.8 from CF₂ fluorine, which is overlapped with spinning sidebands. This is consistent with the structure of the 1H,H-perfluoro-n-octyl acrylate monomer. The spectrum suggests that in vivo studies will require selective excitation due to the different fluorine chemical shifts present in the particles.

Developing fluorinated nanoparticles for multimodal ¹⁹F-MRI and SIMS imaging that possess intrinsic colloidal stability represents a largely unexplored research area. Here, particles less than 500 nm in diameter were synthesized using free radical polymerization, followed by precipitation. Secondary ion mass spectrometry was used to determine the chemical properties of the particles' surface, which will greatly impact their colloidal stability and cytotoxicity. The strong fluorine signal indicated that the particles would be suitable for SIMS imaging applications. It is currently believed that the inclusion of hydrophilic monomers in the polymerization process enhances their colloidal stability and helps prevent irreversible agglomeration that might otherwise occur due to the fluorinated groups.

### Example 2

Materials: All materials were purchased from Sigma-Aldrich (St. Louis, Mo.) unless otherwise stated. 1H,H-perfluoro-n-octyl acrylate was purchased from ExFluor Research Corporation (Round Rock, Tex.). 2-(allyl)hexafluoroisopropanol was purchased from Matrix Scientific (Columbia, S.C.). Vazo-52 was purchased from DuPont (Wilmington, Del.). Dialysis membranes were purchased from Spectrum Labs (Rancho Dominguez, Calif.). Prior to nanoparticle synthesis, (1,5-N-vinylformamido) ethyl ether was synthesized as previously described. Impurities were precipitated out of N-vinylformamide using absolute ethanol prior to use. All other reagents were used as received.

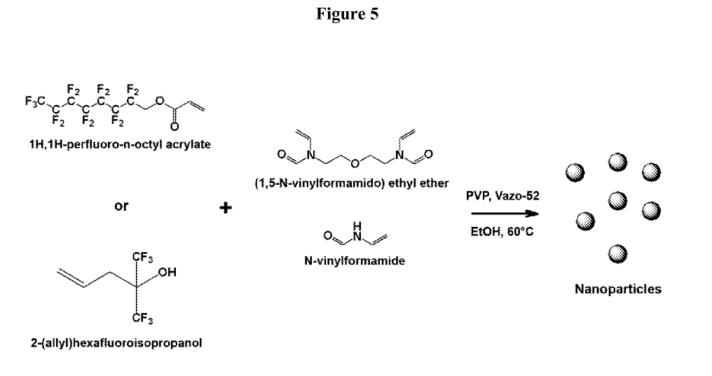

Synthesis of Fluorinated Nanoparticles: Nanoparticles were synthesized using a free radical polymerization technique as described previously. First, 20 μL, of N-vinylformamide, 20 μL, of (1,5-N-vinylformamido) ethyl ether, and 20 μL, of the fluorinated monomer (either 1H,H-perfluoro-n-octyl acrylate, or 2-(allyl)hexafluoroisopropanol) were dissolved in absolute ethanol containing 0.015 mg/mL polyvinylpyrrolidone (PVP) as a surfactant (MW approximately 360 kDa). Next, 6.9 mg of (E)-2,2′-(diazene-1,2-diyl)bis(2,4-dimethylpentanenitrile) (Vazo-52) were added to the solution as an initiator (FIG. 5).

The reagent mixture was then sparged with argon for 10 minutes to remove dissolved oxygen, then heated in a silicone oil bath to 60° C. and stirred at approximately 900 RPM. The reaction was carried out isothermally under an argon atmosphere for 24 hours. The product was then dialyzed against deionized water using a 1 kDa MWCO regenerated cellulose ester dialysis tube (Spectrum Laboratories, Inc., Rancho Dominguez, Calif.) for 24 hours. The dialysate was changed at least 5 times to ensure complete solvent exchange. Linear polymers were synthesized analogously, but without the addition of (1,5,-N-vinylformamido) ethyl ether crosslinker or PVP surfactant. Particles were then further purified by centrifugation for 1 hour at 18,000 rpm. Each centrifugation cycle was repeated at least 3 times.

For the hydrolysis step, particle and polymer suspensions were flash-frozen in liquid nitrogen and then lyophilized. Samples were resuspended in 1 M NaOH_{(aq)} and sonicated for 1 hour at ambient temperature, as described previously. The pH of each solution was then titrated to approximately 7 using 1 M HCl_{(aq)}, and the suspensions were dialyzed against deionized water (MWCO 100 Da) for at least 24 hours to remove salt and hydrolysis byproducts. Dialysate was changed at least 3 times. The hydrolyzed particles and polymers were then flash-frozen in liquid nitrogen and lyophilized prior to analysis with FTIR.

Neutron Scattering: SANS experiments were carried out on the NG7 30 m SANS instrument at the NIST Center for Neutron Research (NCNR) in Gaithersburg, Md. Neutrons of wavelength λ=6 Å with a distribution of Δλ/λ=10% were incident on samples held in quartz cells. Three different sample to detector distances were used to give an overall q range of 0.0038 Å<q<0.37 Å, where q=(4π/λ)sin(θ/2) is the magnitude of the scattering vector. Sample scattering was corrected for background and empty cell scattering, and the sensitivities of individual detector elements were normalized. The corrected data sets were circularly averaged and placed on an absolute scale using software supplied by the NCNR.

USANS experiments were carried out on the BT54 perfect crystal diffractometer (PCD) at the NCNR using the same samples described above. The overall q range was 3.8×10⁻⁵ Å<q<0.002 Å. All data were reduced and analyzed using software provided by the NCNR. Here, only slit-smeared data are shown.

Samples for all neutron scattering experiments were prepared using D₂O as a solvent, thus the scattering length density of the solvent was 6.3×10⁻⁶ Å⁻². Particle densities were approximated using a weighted average of the neat monomer densities. Scattering length densities of the particles were calculated using the NCNR scattering length density calculator with atomic scattering lengths obtained from V.F. Sears.

TEM Imaging: TEM images were acquired on a Philips CM300FEG TEM (Cₛ=1.2 mm) operated at 300 kV using a 10.8 mrad objective aperture semiangle. Samples were drop-mounted onto silicon-supported 25 nm thermal SiO₂ membranes (Dune Sciences NG01-011A) and allowed to dry in air for 24 hours before imaging. Samples were imaged using a Gatan GIF-200 in energy filtered mode using a 10 eV acceptance slit. To increase signal to noise ratios, the images were constructed from a series of 100, 0.5 second exposures, which were then aligned and summed to produce the final image.

Fourier Transform Infrared Spectroscopy: FTIR was used to determine the effects of hydrolysis on the functional groups present within the nanoparticles (Smiths Illuminate FTIR Microscope). After lyophilization, hydrolyzed and non-hydrolyzed samples were resuspended in deionized water by sonicating in an ultrasonication bath for one hour. Particle and polymer suspensions were then decanted onto a gold-plated microscope slide and allowed to dry at ambient conditions under a chemical hood. FTIR spectra were then collected from the resultant polymer and particle films. All experiments were conducted on a diamond attenuated total reflectance objective microscope accessory. Reported spectra are the average of 128 scans.

¹⁹F-NMR Spectroscopy: ¹⁹F spectra were acquired on a Bruker DRX 400 MHz NMR equipped with a QNP probe at The University of Kansas. The samples were suspended in D₂O and the spectrometer was locked to this solvent during the experiments. All samples were recorded with a sweep width of 99 ppm, a transmitter offset of −80 ppm and a 1 second delay. Spectra were collected with 16 scans on the particles synthesized with the 1H,H-perfluoro-n-octyl acrylate monomer and 64 scans were used for the particles synthesized using the 2-(allyl)hexafluoroisopropanol monomer.

Results

Small Angle Neutron Scattering: Fluorinated nanoparticles were prepared using a method similar to one described previously. After purification, samples were suspended in D₂O and analyzed using SANS and USANS at several different concentrations. The scattering length density values for particles prepared with each of the fluorinated monomers are given in Table 1 below.

TABLE 1

| | Estimated Particle | Scattering Length |
|---|---|---|
| Monomer | Density (g/cm3) | Density (Å-2) |
| 1H,1H-perfluoro-n-octyl | 1.30 | 3.00 × 10−6 |
| acrylate | | |
| 2-Allyl | 1.15 | 2.73 × 10−6 |
| hexafluoroisopropanol | | |

Non-linear least-squares model fitting was performed using Schulz-distributed spheres of uniform scattering length density, given by the normalized Schulz distribution:

$$f ( R ) = \frac{{( z + 1 )}^{( z + 1 )} {x}^{z}}{{R}_{avg} Γ ( z + 1 )} exp [ - ( z + 1 ) x ]$$

where R_{avg} is the mean radius of the particles, and x=R/R_{avg}, z is a function of the polydispersity, p=σ/R_{avg}, and z=1/p₂−1, σ is the standard deviation of the distribution, and Γ(z+1) is the gamma function. For a typical SANS experiment, the SANS intensity is given by:

I(q)=φVₚΔρ²P(q)S(q)+b

where φ is the particle volume fraction, Vₚ is the particle volume, Δρ is the difference between the scattering length density of the particles and the scattering length density of the solvent (ρₚ−ρₛ), and b is the incoherent background scattering intensity. The function P(q) is the intraparticle form factor averaged over the Schulz distribution, and S(q) is the interparticle interference factor, which is equal to 1 in the case of non-interacting particles. The particles were confirmed to be non-interacting by measuring at several dilutions. Combining this equation with the normalized Schulz distribution yields:

$$I ( q ) = {( \frac{4 π}{3} )}^{2} ( \frac{φ}{〈 V 〉} ) Δ {ρ}^{2} {\int }_{0}^{∞} f ( R ) {R}^{6} {F}^{2} ( qR ) d R$$

where, <V> is the average volume of a particle. The function F is the scattering amplitude of a sphere, and is given by:

$$F ( x ) = \frac{[ sin ( x ) - x cos ( x ) ]}{{x}^{3}}$$

The average volume, <V>, is calculated using the 3^{rd} moment of R and is given by:

$$〈 V 〉 = \frac{4 π}{3} {( {R}_{avg} )}^{3} \frac{( z + 3 ) ( z + 2 )}{{( z + 1 )}^{2}}$$

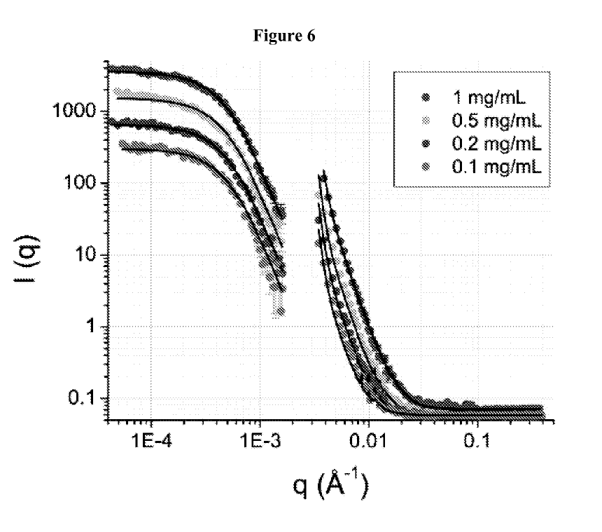

For the particles prepared using the 1H,1H-perfluoro-n-octyl acrylate monomer, the USANS data were initially fit to obtain the size and polydispersity of the particles. For the smaller concentrations (concentrations less than 1 mg/mL), the polydispersity was held at the value calculated from the most concentrated sample. The SANS data were also fit using the parameters calculated from the USANS data, although the background and the volume fraction parameters were relaxed. The model was then fit to both the USANS and the SANS data simultaneously to optimize the fit. The spectra are shown in FIG. 6, and the model parameters are listed in Table 2. The model suggests that the particles are between 390.1 nm±4.4 nm and 413.1 nm±2.6 nm in diameter with a polydispersity of 0.5218. The discrepancies between the volume fractions calculated for the USANS and SANS data for each concentration could be due to small amounts of separation that occurred while the samples were waiting for analysis on the SANS instrument.

TABLE 2
Model parameters for nanoparticles prepared with 1H,1H-perfluoro-n-octyl
acrylate monomer

| Parameter | SANS | USANS | SANS | USANS |
|---|---|---|---|---|
| | 1 mg/mL | | 0.5 mg/mL | |
| Volume Fraction | 2.99 × 10⁻³ ± 2.8 × 10⁻⁵ | 1.32 × 10⁻³ ± 1.4 × 10⁻⁵ | 1.30 × 10⁻³ ± 1.3 × 10⁻⁵ | 3.54 × 10⁻⁴ ± 4.1 × 10⁻⁶ |
| Mean Diameter | 413.1 ± 2.6 | | 410.6 ± 2.8 | |
| (nm) | | | | |
| Polydispersity | 0.522 | | 0.522 | |
| Background (cm⁻¹ | 0.071 | | 0.057 | |
| sr⁻¹) | | | | |
| | 0.2 mg/mL | | 0.1 mg/mL | |
| Volume Fraction | 5.92 × 10⁻⁴ ± 6.5 × 10⁻⁶ | 1.41 × 10⁻⁴ ± 1.9 × 10⁻⁶ | 2.88 × 10⁻⁴ ± 4.8 × 10⁻⁶ | 6.03 × 10⁻⁵ ± 1.1 × 10⁻⁶ |

| | | |
|---|---|---|
| Mean Diameter | 399.1 ± 3.0 | 390.1 ± 4.4 |
| (nm) | | |
| Polydispersity | 0.522 | 0.522 |
| Background (cm⁻¹ | 0.056 | 0.059 |
| sr⁻¹) | | |

Error is equal to 1 standard deviation of the fitted value. Values without reported error were held exact during model convergence.

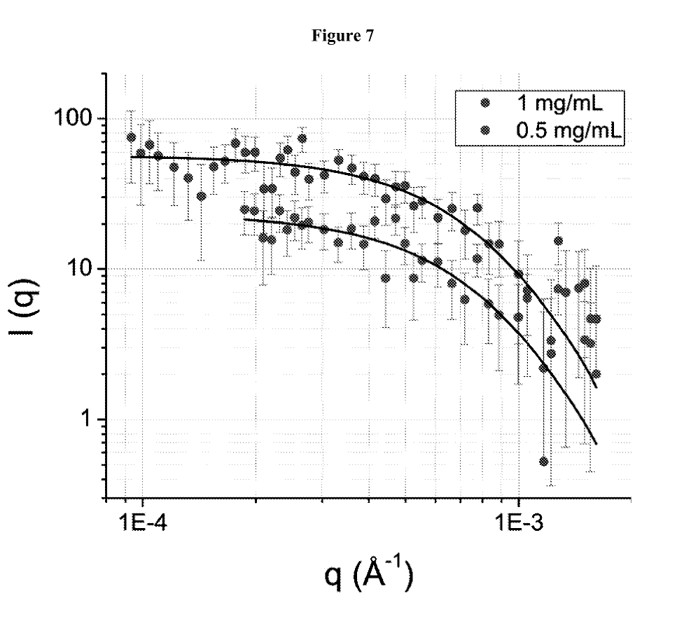

For the 2-(allyl)hexafluoroisopropanol monomer, only spectra obtained for concentrations of 1 mg/mL and 0.5 mg/mL were analyzed due to insufficient scattering at lower concentrations. Only the USANS spectra were used in the analysis of 2-(allyl)hexafluoroisopropanol because the SANS data were too noisy and the expected particle size was within the range suitable for USANS measurements. The model was fit to each data set individually, and then to both concentrations simultaneously while holding the polydispersity from the highest concentrated sample constant to minimize the degrees of freedom and to ensure a consistent fit with both data sets. In the spectra for the particles prepared with the 2-(allyl)hexafluoroisopropanol monomer, the data were too noisy to accurately determine both the particle size and the polydispersity, so the polydispersity was held at 0.5, which is close to the value obtained for the particles prepared with the 1H,H-perfluoro-n-octyl acrylate monomer. The spectra for these particles are shown in FIG. 7, and the model parameters are listed in Table 3. The model suggests an average particle size of 281.2 nm±16.4 nm.

TABLE 3
Model parameters for nanoparticles prepared with 2-allyl
hexafluoroisopropanol monomer

| Parameter | 1 mg/mL | 0.5 mg/mL |
|---|---|---|
| Volume Fraction | 1.62 × 10⁻³ ± 1.5 × 10⁻⁴ | 6.56 × 10⁻⁴ ± 6.2 × 10⁻⁵ |

| | |
|---|---|
| Mean Radius (nm) | 281.2 ± 16.4 |
| Polydispersity | 0.5 |
| Background | 0.218 |
| (cm⁻¹ sr⁻¹) | |

Error is equal to 1 standard deviation of the fitted value. Values without reported error were held exact during model convergence.

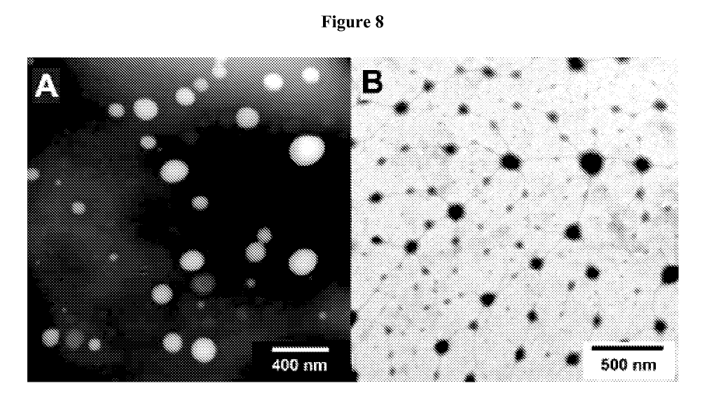

Transmission Electron Microscopy: The sizes of the fluorinated nanoparticles were further corroborated by the TEM images (FIG. 8), which show particles prepared with the 1H,H-perfluoro-n-octyl acrylate monomer (Panel A) that are less than 400 nm in diameter. Panel B shows particles prepared with the 2-(allyl)hexafluoroisopropanol monomer that are less than 500 nm in diameter. The observed particle sizes corroborate the neutron scattering data. This suggests that the particles might be useful as a passive targeting agent for tumor imaging.

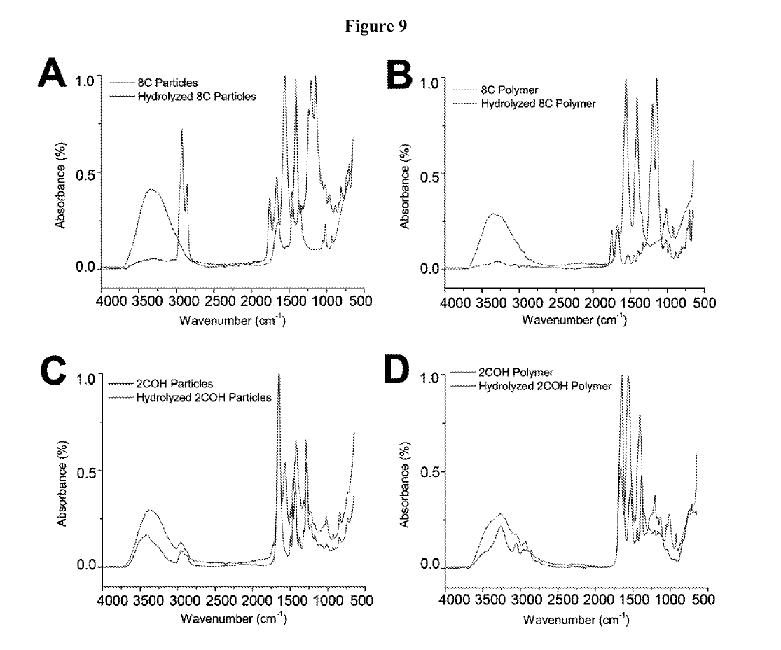

Fourier Transform Infrared Spectroscopy: Nanoparticles were analyzed using FTIR to determine the suitability of the particles for chemical modification and conjugation strategies. Analogous polymers prepared without the (1,5-N-vinylformamido) ethyl ether crosslinker or the PVP surfactant were also analyzed as a comparison. The ability to hydrolyze the amide to an amine could enable one to conjugate a carboxyl terminated targeting ligand to the particles or polymers to facilitate active targeting of pathological tissue. This can be done with simple chemistry that is well described in the literature. Particles prepared using the 1H,H-perfluoro-n-octyl acrylate monomer (FIG. 9, Panel A) showed evidence of hydrolysis of both the formamide group and of the perfluorinated ester.

Hydrolyzed particles showed a strong peak at 3326 cm⁻¹, which is within the hydrogen bonded OH region (3300-2500 cm⁻¹) and the primary aliphatic amine region (3450-3250 cm⁻¹). The emergence of the OH frequency was due to the hydrolysis of the perfluorinated acrylate (1H,H-perfluoro-n-octyl acrylate), which was hydrolyzed to a carboxylic acid. Although the presence of an amine after hydrolysis suggests that the particles can be bound to a targeting ligand using simple chemistry, the fact that the fluorinated ester was hydrolyzed indicates that particles prepared with the 1H,H-perfluoro-n-octyl acrylate monomer would not be suitable for use as an active targeting agent if conversion to the primary amine is necessary. There was no evidence of ether hydrolysis.

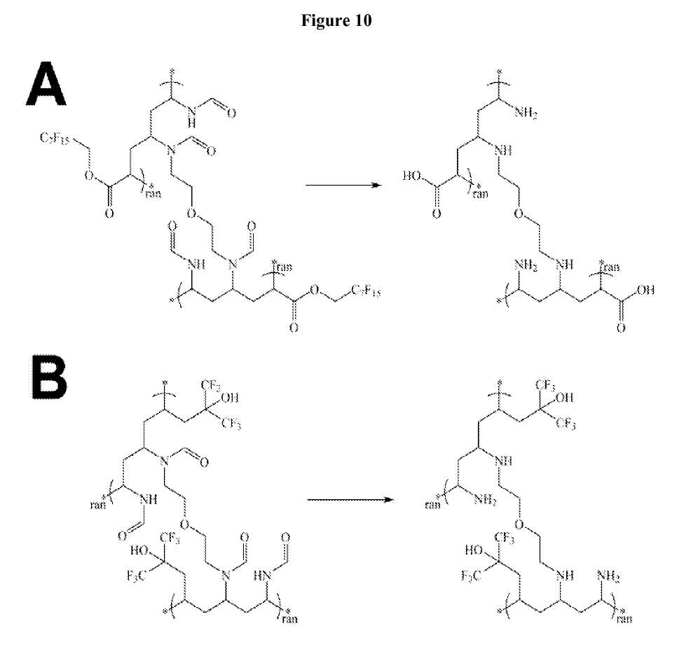

The non-hydrolyzed particles prepared with the 1H,H-perfluoro-n-octyl acrylate showed a peak at 1753 cm⁻¹ suggesting a carbonyl ester (1750-1725 cm⁻¹) and an amide I carbonyl peak (1670-1650 cm⁻¹), both of which were absent in the hydrolyzed particles' spectrum. The hydrolyzed particles also showed an N—H deformation peak at 1548 cm⁻¹, which is near the typical region (1650-1580 cm⁻¹) but was likely shifted to higher frequency due to hydrogen bonding. This would indicate the presence of an amine. Representative structures of the non-hydrolyzed and hydrolyzed particles are shown in FIG. 10.

The analogous polymer samples (FIG. 9, Panel B) synthesized with the 1H,H-perfluoro-n-octyl acrylate monomer showed much evidence of hydrolysis. The hydrolyzed polymer spectrum showed a broad peak around 3300 cm₋₁, which is indicative of both OH stretching (3300-2500 cm⁻¹) and primary amines (3450-3250 cm⁻¹). The non-hydrolyzed spectrum showed small peaks at 1749 cm⁻¹ and 1669 cm⁻¹, both of which were absent in the hydrolyzed spectrum and indicated the presence of a carbonyl ester (1750-1725 cm⁻¹) and an amide I (carbonyl) peak (1670-1650 cm₋₁), respectively. Additionally, two peaks at 1196 cm⁻¹ and 1140 cm⁻¹ were present in the non-hydrolyzed spectrum, and indicated the presence of an ester C—O stretch. These peaks were very obviously absent in the hydrolyzed spectrum, which suggested the hydrolysis of the fluorinated ester group.

Nanoparticles prepared using the 2-(allyl)hexafluoroisopropanol monomer (FIG. 9, Panel C) qualitatively suggested hydrolysis of the formamide group on the N-vinylformamide monomer and the (1,5-N-vinylformamido) ethyl ether crosslinker. The non-hydrolyzed spectrum showed a peak at 3401 cm⁻¹, which is within the OH stretch region (3300-2500 cm⁻¹). Unfortunately, this overlaped with the primary amine region (3450-3250 cm⁻¹), making it difficult to distinguish the two. The hydrolyzed particles showed a broad peak at 3378 cm⁻¹. Because this frequency is shifted toward the primary amine region and is at higher intensity, it stands to reason that this peak was due to influence from both the hydroxyl group on the 2-(allyl)hexafluoroisopropanol monomer and the primary amine from the hydrolyzed formamide groups. Additionally, both spectra showed peaks at 1648 cm⁻¹, which suggested the presence of a hydrogen-bonded carbonyl group. This is most likely from the formamide group (amide I), which was present in the non-hydrolyzed particles and was likely present within the hydrolyzed particles to some degree, possibly buried within the particles where they might be sterically shielded from hydrolysis. The high intensity of this peak is not observed in the linear hydrolyzed polymer, which suggested that the crosslinking may have buried some of the formamide groups within the particles. The presence of an amine after hydrolysis suggested that these particles could be successfully conjugated with a carboxyl terminated targeting ligand. Representative structures of the hydrolyzed and non-hydrolyzed particles are shown in FIG. 3.6. Like in the previous sample, there was no evidence of ether hydrolysis.

The spectra for the analogous polymer samples (FIG. 9, Panel D) show evidence of formamide hydrolysis. Like the particles, both the hydrolyzed and non-hydrolyzed polymers show peaks in the OH stretch region (3300-2500 cm⁻¹), which is expected from to the 2-(allyl)hexafluoroisopropanol monomer. This region overlaps with the primary aliphatic amine region (3450-3250 cm⁻¹) and is more intense for the hydrolyzed polymer, suggesting that this peak is influenced by both the hydroxyl group and the primary amine. Additional evidence for hydrolysis is the decrease in the intensity of the carbonyl peak (1650 cm⁻¹), which is likely shifted to a higher frequency in both spectra due to hydrogen bonding. The lower intensity of this peak in the hydrolyzed sample could indicate the loss of the formamide group, which would be expected. The fact that this peak did not diminish in intensity in the particles could be due to the crosslinking, which might bury formamide groups within the particles, again rendering them sterically isolated from hydrolysis.

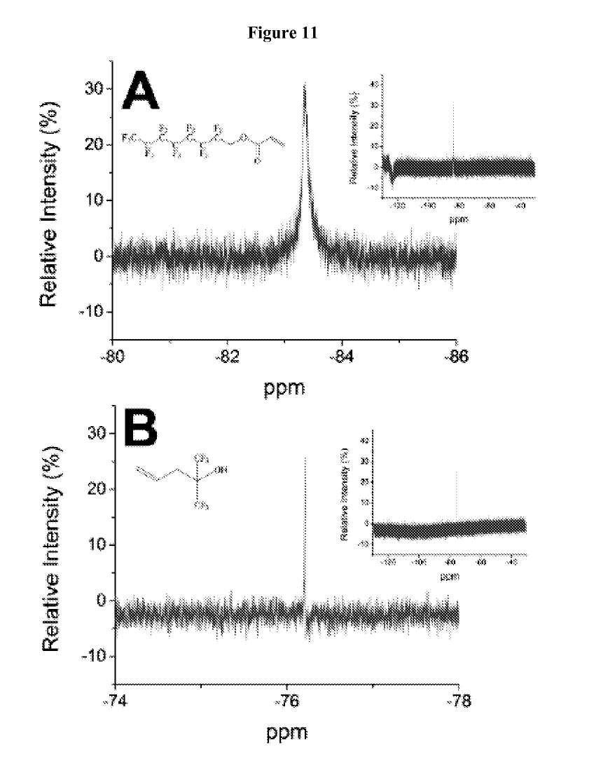

¹⁹F NMR Spectroscopy: Nanoparticle samples were analyzed using ¹⁹F-NMR to determine their potential as ¹⁹F-MRI contrast agents. FIG. 11 shows the ¹⁹F-NMR spectra of the fluorinated nanoparticles.

Particles prepared with the 1H,H-perfluoro-n-octyl acrylate monomer showed a peak between −83 ppm and −84 ppm (FIG. 11, Panel A). This peak is from the CF₂ group closest to the oxygen atom. Additionally, the spectrum shows an out-of-phase signal near −121 ppm (inset), which is from the perfluorinated region (—CF₂CF₂—) of the pendant group. Particles prepared with the 2-(allyl)hexafluoroisopropanol monomer show a peak around −76 ppm (FIG. 11, Panel B). This peak is similar to the CF₂ peak from the 1H,H-perfluoro-n-octyl acrylate particles because both have the same proximity to a nearby oxygen atom. Both particles exhibit fairly narrow widths in their ¹⁹F-NMR spectra, suggesting that some fluorinated groups are well solvated with a degree of mobility. This suggests that the particles would produce a signal in ¹⁹F-MRI and would therefore be suitable for use as a ¹⁹F-MRI contrast agent.

### Example 3

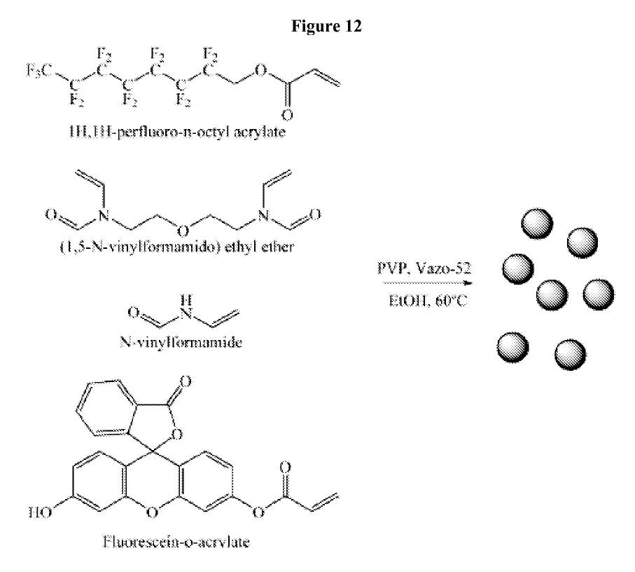

In this example, fluorinated-fluorescent nanoparticles were synthesized and evaluated as a multimodal in vitro imaging probe for optical fluorescence and SIMS imaging (FIG. 12).

Materials: All materials were purchased from Sigma-Aldrich unless otherwise stated. 1H,H-perfluoro-n-octyl acrylate was purchased from ExFluor Research Corporation (Round Rock, Tex.). (E)-2,2′-(diazine-1,2-diyl)bis(2,4-dimethylpentanenitrile) (Vazo-52) was purchased from DuPont (Wilmington, Del.). Dialysis membranes were purchased from Spectrum Labs (Rancho Dominguez, Calif.). Prior to nanoparticle synthesis, (1,5-N-vinylformamido) ethyl ether was synthesized as previously described. Impurities were precipitated out of N-vinylformamide using absolute ethanol and vacuum filtered prior to use. All other reagents were used as received.

Fluorinated-Fluorescent Nanoparticle Synthesis: Nanoparticles (NPs) were synthesized using a free radical polymerization method similar to one described previously. First, 20 μL of 1H,H-perfluoro-n-octyl acrylate, 20 μL of (1,5-N-vinylformamido) ethyl ether and 20 μL of N-vinyl formamide were dissolved in absolute ethanol containing 0.015 g/mL polyvinylpyrrolidone (PVP) as a surfactant (MW approximately 360 kDa). Next, 0.0055 mg of fluorescein-o-acrylate and 0.0076 mg of Vazo-52 initiator were added to the solution under stirring. The reagent mixture was sparged with nitrogen for 10 minutes to remove dissolved oxygen, then was heated in a silicone oil bath to 60° C. and stirred. The reaction was carried out isothermally under a nitrogen atmosphere for 24 hours. The reaction vessel was protected from ambient light to minimize photobleaching of the fluorescent monomer. The product was then dialyzed against deionized water using a 500 Da MWCO regenerated cellulose ester dialysis tube for 24 hours. The dialysate was changed at least 5 times to ensure complete solvent exchange and the removal of unreacted fluorescein-o-acrylate monomer. The resultant nanoparticle suspension was then purified by centrifugation for 1 hour at 18,000 rpm. Each centrifugation cycle was repeated at least 3 times. Particles were then flash-frozen in liquid nitrogen and lyophilized. After lyophilization, resuspended particle sizes and zeta potentials were measured using dynamic light scattering.

Aminooxylated LABL Peptide Synthesis: Aminooxy peptides were synthesized using 9-fluorenylmethyloxycarbonyl-protected amino acid chemistry on polyethylene glycol-polystyrene resins. The peptides synthesized where aminooxy LABL (aminooxy-ITDGEATDSG), an ICAM-1 antagonist. Peptides were deprotected, cleaved from resin, and isolated by precipitation in ether. Purification was completed using preparatory high performance liquid chromatography (HPLC), followed by lyophilization. Peptide identity was verified and purity/content was assessed using analytical HPLC and mass spectroscopy.

Conjugation of Aminooxylated LABL Peptide to NPs: For the conjugation step, 5.9 mg of nanoparticles were re-suspended in 5.9 mL of 20 mM acetate buffer, to a final concentration of 1 mg/mL. Particles were then sonicated for 10 minutes to disperse the suspension. A volume of 3 mL (approximately 3 mg of nanoparticles) was transferred to a separate reaction flask, to which 21.43 mg of aminooxy-LABL (aminooxy-ITDGEATDSG) was added and dissolved by stirring. The pH of both the nanoparticle (NP) solution and LABL-conjugated NP solution (LABL-NPs) was measured and adjusted to pH 5.5. Reaction flasks were stirred at 500 RPM for 16 hours. Reaction time was based on previously conducted studies (unpublished data). After the reaction, the solution was extensively dialyzed against deionized H₂O (MWCO 3500 Da) to remove unreacted peptide, followed by lyophilization of the dialyzed product. Conjugation efficiency was measured using HPLC.

Up-regulation of ICAM-1 by Tumor Necrosis Factor-α: HUVEC cells (4.5×10⁵ cells in 80 μl of serum free F12K medium) were stimulated using 1,000 U/ml of Tumor Necrosis Factor-α (TNF-α) for 24 hrs. Cells at the same concentration were not activated and used as a control. HUVEC cells, with or without ICAM-1 upregulation, were incubated with 5% BSA in PBS for 10 mM at 4° C. and then anti-ICAM-1-FITC (0.05 mg/ml) was added to cells and incubated at 4° C. for 45 mM. Free antibodies were removed by rinsing three times with PBS after centrifugation (4,000 RPM, 3 min). The fluorescence intensity of the cells was analyzed by flow cytometry. Data analysis was performed using Cell Quest software (BD).

Binding and Uptake of LABL-NPs by HUVEC Cells: The binding and uptake of LABL-NPs was studied using fluorescence spectroscopy. TNF-α stimulated HUVEC cells (5×10⁵ cells/ml) were added to a 96 well-plate (100 μl/well) and incubated with LABL-NPs or NPs (3.7 mg/ml, 30 μl) at 37° C. for 5, 15, 30 and 60 min and washed with PBS. The fluorescence intensity of cells was measured using a fluorescence plate reader (Spectramax M5; ex., 450 nm; em., 500 nm).

Fluorescence Microscopy of the Uptake of LABL-NPs by HUVEC Cells: Fluorescence microscopy was used to compare the extent of binding and uptake of LABL-NPs and unconjugated NPs in HUVEC cells. HUVEC cells (2.5×10⁵ cells/ml) were activated by using 1,000 U/ml TNF-α for 24 hrs. Cells were then added to an 8-well plate. LABL-NPs or unconjugated fluorescent NPs (2.5 mg/ml) were incubated with the cells for 5, 15, 30 min and 1 hr at 37° C., 5% CO₂. Unbound nanoparticles were removed by washing three times with PBS and fixed with 4% paraformaldehyde. Nuclei were labeled with DAPI dilactate (blue) (300 nM, ex: 358 nm, em: 461 nm) for 5 min at 37° C., 5% CO₂. Fluorescence micrographs were acquired using the UV and FITC filter sets of a Nikon Eclipse 80i microscope equipped for epifluorescence. Micrographs were captured using an Orca ER camera (Hamamatsu, Inc., Bridgewater, N.J.) and analyzed by Metamorph, version 6.2 (Universal Imaging Corp., West Chester, Pa.).

Lysosomal Trafficking of LABL-NPs in HUVEC Cells: Fluorescence microscopy was utilized to investigate the intracellular fate of LABL-NPs and untargeted NPs in HUVEC cells. HUVEC cells (2.5×10⁵ cells/ml) were activated with 1,000 U/ml of TNF-α for 24 hrs. Cells were incubated with Texas red dextran (Mw 10,000, lysine fixable, 1 mg/ml) for 2 hours at 37° C., 5% CO₂ and washed three times with PBS. Cells were then incubated in serum free media at 37° C., 5% CO₂ for 12 hrs to allow the dye to reach the lysosomes. Afterward, LABL-NPs or unconjugated NPs (200 μl, 2.5 mg/ml) were added to cells and incubated for 5, 15, 30 and 60 min at 37° C., 5% CO₂. Unbound nanoparticles were removed by washing three times with PBS. Nuclei were labeled with DAPI dilactate (blue) (300 nM, ex: 358 nm, em: 461 nm) for 5 min at 37° C., 5% CO₂. Cells were then fixed with 4% paraformaldehyde. Fluorescence emissions of nanoparticles and lysosomes were observed using FITC and rhodamine filter sets, respectively (Nikon Eclipse 80i microscope equipped for epifluorescence). Micrographs were captured using an Orca ER camera (Hamamatsu, Inc.). Colocalization of nanoparticles with lysosomes was analyzed by Metamorph, version 6.2.

Development of Cell Sample Prep Method for SIMS Imaging: A cell sample preparation method was designed for SIMS imaging of nanoparticles in cells. A polished silicon disk was wiped clean several times with an ethanol-dampened, lint-free cloth. Residual dust was removed with pressurized nitrogen. The disk was placed into a 35-mm petri dish and covered with a fibronectin solution (25 ug/mL, Sigma) in PBS. After 3 hours at 4° C., the disk was removed, rinsed with PBS and placed in a 35-mm petri dish. A10 rat aorta smooth muscle cells (ATCC) maintained in DMEM containing 10% FBS at 37° C. in a 5% CO₂ incubator were seeded onto the disks at density of 1200 cells/cm². The disks were placed back into the incubator for 48 hours. The samples were then rinsed in PBS and fixed in 1% formaldehyde in PBS for 4 hours. The samples were rinsed in PBS, then DI H₂O and were allowed to dry in the laminar hood for 4 hours.

TOF-SIMS (time of flight secondary ion mass spectrometry) was used to analyze the cell samples. TOF-SIMS experiments were performed on an Ion-TOF IV instrument equipped with both Bi (Biₙ⁺, where n=1 to 7) and SF₅⁺ primary ion beam cluster sources. The analysis source was a pulsed, 25 keV bismuth cluster ion source (Bi₃⁺), which bombarded the surface at an incident angle of 45 degrees to the surface normal. The target current was maintained at approximately 0.3 pA (±10%) pulsed current with a raster size of 200 μm×200 μm for all experiments. Both positive and negative secondary ions were extracted from the sample into a reflectron-type time of flight mass spectrometer. The secondary ions were then detected by a microchannel plate detector with a post-acceleration energy of 10 kV. A low energy electron flood gun was utilized for charge neutralization in the analysis mode. Each spectrum was averaged over a 60 s time period, with a cycle time of 100 μs. These conditions resulted in accumulated Bi3+ ion doses that were well below 10¹³ ions/cm².

Statistical Analysis: Statistical evaluation of data was performed using an analysis of variance (single-factor ANOVA). Tukey's test was used as a post hoc analysis to assess the significance of differences. A value of p<0.05 was accepted as significant.

Results and Discussion

Preparation of Fluorinated-Fluorescent Nanoparticles: Fluorinated-fluorescent nanoparticles were prepared using a free radical polymerization method, similar to what has been described previously. Nanoparticles were conjugated with aminooxy-LABL peptide using an oxime formation strategy, resulting in a conjugation efficiency of 93.8%±0.05 as measured by HPLC. Dynamic light scattering showed a mean particle diameter of 440 nm±4.3 nm for the unconjugated NPs and 354 nm±10 nm for the LABL-NPs (Table 4).

TABLE 4
Dynamic light scattering and zeta potential data for Nanoparticles

| | | | Zeta Potential |
|---|---|---|---|
| | Diameter (nm) | Polydispersity | (mV) |
| NP | 440 ± 4.3 | 0.21 ± 0.019 | −5.08 ± 0.86 |
| LABL-NP | 354 ± 10 | 0.167 ± 0.083 | −10.03 ± 3.27 |

The polydispersities for the particle samples were 0.21±0.019 for the unconjugated NPs and 0.167±0.083 for the LABL-NPs, and the measured zeta potentials were −5.08 mV±0.86 mV and −10.03 mV±3.27 mV for the unconjugated NPs and the LABL-NPs, respectively. The observed decrease in NP size after conjugation with the LABL peptide could be due to increased colloidal stability arising after conjugation due to the increased surface charge magnitude, which results from the presence of anionic amino acid residues in the LABL peptide. Fluorinated groups on the NPs surface would be extremely hydrophobic, which might cause agglomeration, and hence an increased observed particle size for the unconjugated NPs. Presumably, these hydrophobic interactions are mitigated by the presence of the LABL peptide, which decreases the tendency of the NPs to agglomerate and hence the observed particle size.

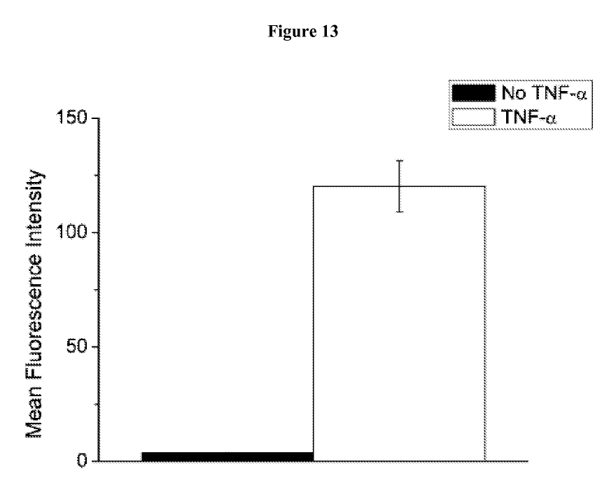

Binding and uptake of nanoparticles in cells: Proinflammatory cytokines such as TNF-α have previously been shown to upregulate the expression if ICAM-1. HUVEC cells were incubated with 1,000 U/ml of TNF-α for 24 hrs to induce overexpression of ICAM-1. HUVEC cells, with or without ICAM-1 upregulation, were incubated with anti-ICAM-1-FITC, which resulted in an approximately 32×increase in ICAM-1 expression compared to HUVEC cells incubated in medium without TNF-α. The result confirmed the overexpression of ICAM-1 and validated the use of this cell line for this study (FIG. 13).

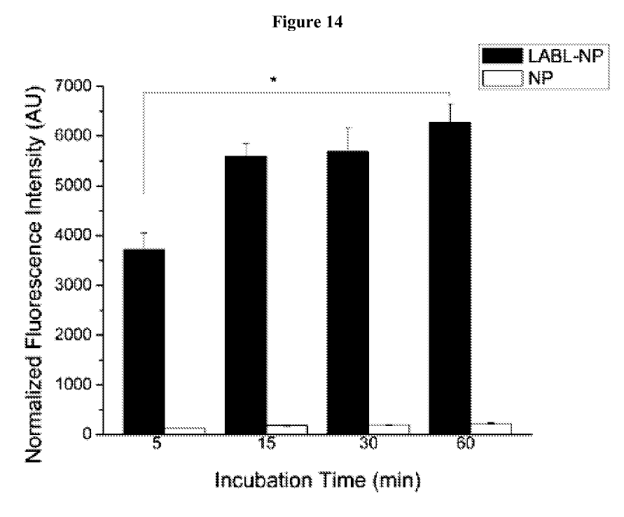

Nanoparticles conjugated with LABL peptide were rapidly taken up by HUVEC cells, as determined by fluorescence measurements after incubation for several time points (FIG. 14).

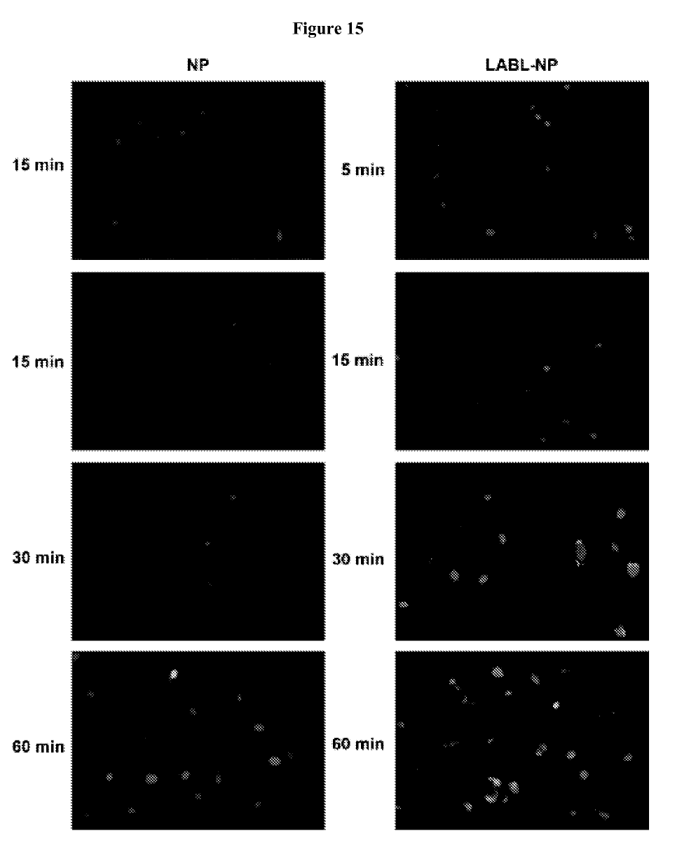

The normalized fluorescence intensity of the LABL-NPs was approximately 30 times greater after any incubation time (5 min to 60 min) than the normalized fluorescence intensity of the non-conjugated NPs, most likely due to binding to ICAM-1, which was facilitated by the LABL peptide. The enhanced binding of nanoparticles to ICAM-1 mediated through LABL and similar peptides has been described by others. 72, 96 Optical images showing fluorescence at several different time points corroborate the quantitative fluorescence measurements (FIG. 15), where nanoparticles (green) can be seen around cell nuclei (blue).

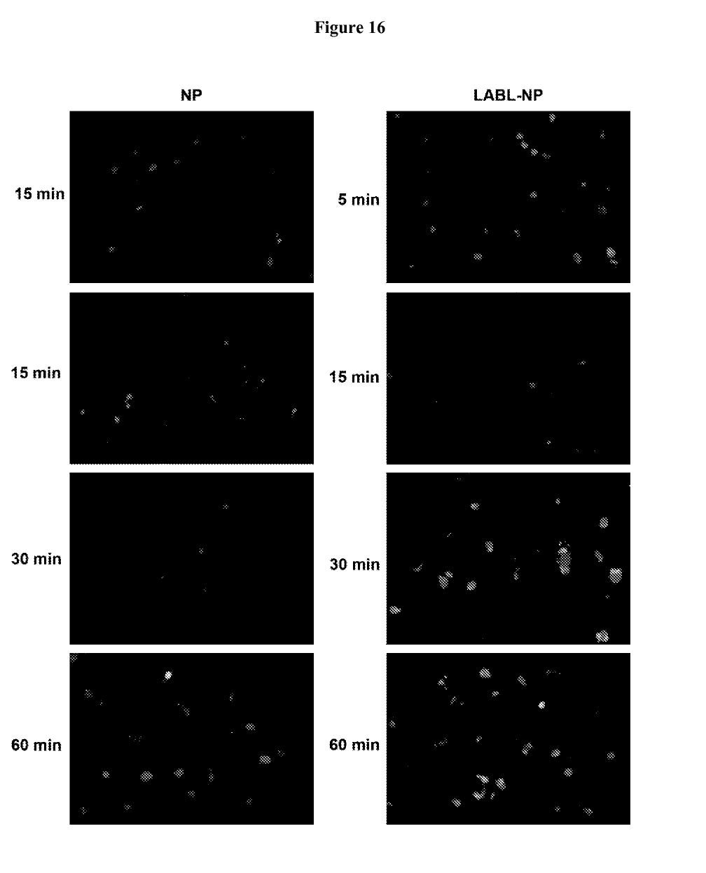

Colocalization of NPs with lysosomes was analyzed using fluorescence imaging (FIG. 16). Lysosomes are labeled red and NPs appear green. The results suggested that neither the LABL-conjugated NPs, nor the non-conjugated NPs, were trafficked to lysosomes, which would appear yellow in the image. This could be due to the large size of the nanoparticles (approximately 300 nm), which might be too large for endocytosis and/or lysosome trafficking. Alternatively, particles could be bound to the surface of cells. This result could be confirmed with SIMS analysis, which will analyze the surface of the cells and the internal composition of the cells by depth profiling.

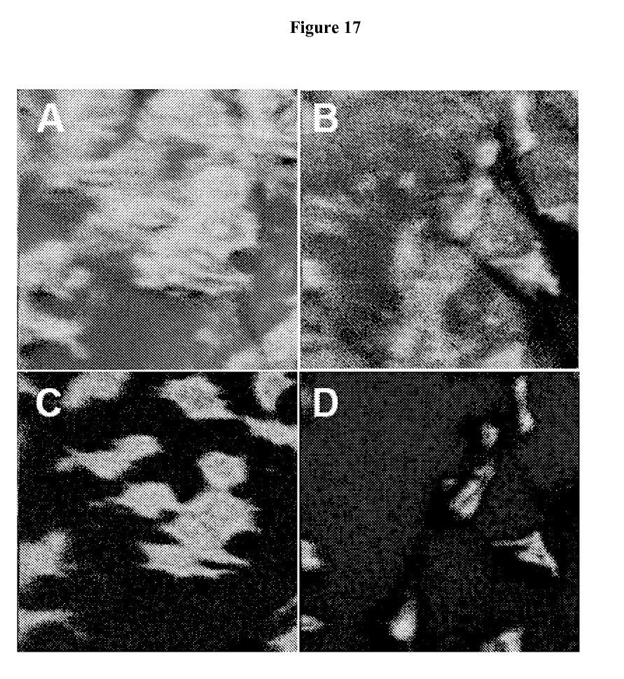

Cell Sample Preparation for SIMS Imaging: A sample preparation method for SIMS imaging of cells was developed using A10 rat aorta smooth muscle cells as a model cell line. Fixed A10 cells on silicon disks were imaged using a TOF-SIMS instrument equipped with an SF₅ sputter source and a Bi₃⁺ analysis source. Cells were distinguishable before sputtering, but the surface contained many ions that would suggest the presence of residual salts, amino acids and proteins deposited from the growth medium and during substrate preparation (FIG. 17). Images acquired after sputtering show less deposited material and greater contrast between the cells and the surrounding substrate. Amines, phosphates, and cholesterol are resolvable on the cell surfaces. The results suggest that this sample preparation method is suitable for producing high-resolution SIMS images of eukaryotic cells and may have applications in biomedical imaging. By incubating cells with an imaging probe that targets a specific cell surface antigen of interest; this technique could be used for in vitro diagnostic applications.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

[1] W. J. Du, A. M. Nystrom, L. Zhang, K. T. Powell, Y. L. Li, C. Cheng, S. A. Wickline, K. L. Wooley, Biomacromolecules 2008, 9, 2826.
[2] F. Duconge, T. Pons, C. Pestourie, L. Herin, B. Theze, K. Gombert, B. Mahler, F. Hinnen, B. Kuhnast, F. Dolle, B. Dubertret, B. Tavitian, Bioconjugate Chemistry 2008, 19, 1921.
[3] M. Huang, Z. L. Huang, M. Bilgen, C. Berkland, Nanomedicine 2008, 4, 30.
[4] J. M. Janjic, M. Srinivas, D. K. K. Kadayakkara, E. T. Ahrens, Journal of the American Chemical Society 2008, 130, 2832.
[5] H. Peng, I. Blakey, B. Dargaville, F. Rasoul, S. Rose, A. K. Whittaker, Biomacromolecules 2009, 10, 374.
[6] W. J. Rieter, K. M. L. Taylor, H. Y. An, W. L. Lin, W. B. Lin, Journal of the American Chemical Society 2006, 128, 9024.
[7] P. Sharma, S.C. Brown, N. Bengtsson, Q. Z. Zhang, G. A. Walter, S. R. Grobmyer, S. Santra, H. B. Jiang, E. W. Scott, B. M. Moudgil, Chemistry of Materials 2008, 20, 6087.
[8] N. K. Devaraj, E. J. Keliher, G. M. Thurber, M. Nahrendorf, R. Weissleder, Bioconjugate Chemistry 2009, 20, 397.
[9] P. D. Piehowski, A. J. Carado, M. E. Kurczy, S. G. Ostrowski, M. L. Heien, N. Winograd, A. G. Ewing, Analytical Chemistry 2008, 80, 8662.
[10] A. F. M. Altelaar, J. van Minnen, C. R. Jimenez, R. M. A. Heeren, S. R. Piersma, Analytical Chemistry 2005, 77, 735.
[11] M. J. Baker, L. Zheng, N. Winograd, N. P. Lockyer, J. C. Vickerman, Langmuir 2008, 24, 11803.
[12] T. L. Colliver, C. L. Brummel, M. L. Pacholski, F. D. Swanek, A. G. Ewing, N. Winograd, Analytical Chemistry 1997, 69, 2225.
[13] S. G. Ostrowski, M. E. Kurczy, T. P. Roddy, N. Winograd, A. G. Ewing, Analytical Chemistry 2007, 79, 3554.
[14] P. D. Piehowski, M. E. Kurczy, D. Willingham, S. Parry, M. L. Heien, N. Winograd, A. G. Ewing, Langmuir 2008, 24, 7906.
[15] T. P. Roddy, D. M. Cannon, S. G. Ostrowski, N. Winograd, A. G. Ewing, Analytical Chemistry 2002, 74, 4020.
[16] K. S. Kulp, E. S. F. Berman, M. G. Knize, D. L. Shattuck, E. J. Nelson, L. G. Wu, J. L. Montgomery, J. S. Felton, K. J. Wu, Analytical Chemistry 2006, 78, 3651.
[17] A. F. M. Altelaar, I. Klinkert, K. Jalink, R. P. J. de Lange, R. A. H. Adan, R. M. A. Heeren, S. R. Piersma, Analytical Chemistry 2006, 78, 734.
[18] Y. P. Kim, E. Oh, M. Y. Hong, D. Lee, M. K. Han, H. K. Shon, D. W. Moon, H. S. Kim, T. G. Lee, Analytical Chemistry 2006, 78, 1913.
[19] R. Schwarz, M. Schuurmans, J. Seelig, B. Kunnecke, Magnetic Resonance in Medicine 1999, 41, 80.
[20] U. Flogel, Z. Ding, H. Hardung, S. Jander, G. Reichmann, C. Jacoby, R. Schubert, J. Schrader, Circulation 2008, 118, 140.
[21] M. Srinivas, P. A. Morel, L. A. Ernst, D. H. Laidlaw, E. T. Ahrens, Magn Reson Med 2007, 58, 725.
[22] I. Brigger, C. Dubernet, P. Couvreur, Advanced Drug Delivery Reviews 2002, 54, 631.
[23] J. B. Matson, R. H. Grubbs, Journal of the American Chemical Society 2008, 130, 6731.
[24] H. Maeda, J. Wu, T. Sawa, Y. Matsumura, K. Hori, Journal of Controlled Release 2000, 65, 271.
[25] X. Yu, S. K. Song, J. Chen, M. J. Scott, R. J. Fuhrhop, C. S. Hall, P. J. Gaffney, S. A. Wickline, G. M. Lanza, Magn Reson Med 2000, 44, 867.
[26] G. W. Buchanan, I. Moudrakovski, Journal of Fluorine Chemistry 2008, 129, 137.
[27] X. B. Fan, J. N. River, A. S. Muresan, C. Popescu, M. Zamora, R. M. Culp, G. S. Karczmar, Physics in Medicine and Biology 2006, 51, 211.
[28] A. M. Neubauer, S. D. Caruthers, F. D. Hockett, T. Cyrus, J. D. Robertson, J. S. Allen, T. D. Williams, R. W. Fuhrhop, G. M. Lanza, S. A. Wickline, J Cardiovasc Magn Reson 2007, 9, 565.
[29] K. C. Partlow, J. Chen, J. A. Brant, A. M. Neubauer, T. E. Meyerrose, M. H. Creer, J. A. Nolta, S. D. Caruthers, G. M. Lanza, S. A. Wickline, Faseb J 2007, 21, 1647.
[30] C. M. Mahoney, A. J. Fahey, Analytical Chemistry 2008, 80, 624.
[31] C. M. Mahoney, D. V. Patwardhan, M. K. McDermott, Applied Surface Science 2006, 252, 6554.
[32] C. M. Mahoney, S. V. Roberson, G. Gillen, Analytical Chemistry 2004, 76, 3199.
[33] V. Tohver, J. E. Smay, A. Braem, P. V. Braun, J. A. Lewis, Proceedings of the National Academy of Sciences of the United States of America 2001, 98, 8950.
[34] F. Zhang, G. G. Long, P. R. Jemian, J. Ilaysky, V. T. Milam, J. A. Lewis, Langmuir 2008, 24, 6504.
[35] J. X. Yu, V. D. Kodibagkar, W. N. Cui, R. P. Mason, Current Medicinal Chemistry 2005, 12, 819.
[36] L. J. Shi, C. Berkland, Advanced Materials 2006, 18, 2315.

What is claimed is:

1. A method comprising:
providing a reaction mixture comprising a fluorinated monomer and a hydrophilic monomer; and
allowing the reaction mixture to polymerize.

2. The method of claim 1, wherein the reaction mixture further comprises a crosslinker.

3. The method of claim 1, wherein the reaction mixture further comprises a surfactant.

4. The method of claim 1, wherein the fluorinated monomer comprises at least one monomer selected from the group consisting of: tetrafluoroethylene, hexafluoropropylene, chlorotrifluoroethylene, trifluoroethylene, hexafluoroisobutylene, perfluoroalkyl ethylene, fluorovinyl ether, vinyl fluoride, vinylidene fluoride, perfluoro-2,2-dimethyl-1,3-dioxole, perfluoro-2-methylene-4-methyl-1,3-dioxolane, perfluoro(allyl vinyl ether), perfluoro(butenyl vinyl ether), and 2-(allyl)hexafluoroisopropanol.

5. The method of claim 1, wherein the fluorinated monomer comprises at least one monomer selected from the group consisting of: 2,3,4,5,6-pentafluorostyrene, 2-fluorostyrene, 3-fluorostyrene, 4-fluorostyrene, 2,6-difluorostyrene, 2-(trifluoromethyl)styrene, 3-(trifluoromethyl)styrene, and 4-(trifluoromethyl)styrene, 2,2,2-trifluoroethyl methacrylate, 1H, 1H-perfluoro-n-octyl acrylate, 1H,1H-perfluoro-n-decyl acrylate, 1H,1H-perfluoro-n-octyl methacrylate, 1H,1H-perfluoro-n-decyl methacrylate, hexafluoroisopropyl acrylate, and hexafluoroisopropyl methacrylate.

6. The method of claim 1, wherein the hydrophilic monomer comprises at least one monomer selected from the group consisting of: N-vinylformamide, acrylates, and polyethylene glycol-acrylates.

7. The method of claim 1, wherein allowing the reaction mixture to polymerize comprises incubating the reaction mixture at about 50° C. for about 24 hours.

8. A method comprising:
providing a nanoparticle composition comprising a fluorinated monomer component and a hydrophilic monomer component;
introducing the nanoparticle composition into a subject; and
obtaining an image of at least a portion of the subject.

9. The method of claim 8, wherein the nanoparticle composition further comprises a crosslinker.

10. The method of claim 8, wherein the fluorinated monomer comprises at least one monomer selected from the group consisting of: tetrafluoroethylene, hexafluoropropylene, chlorotrifluoroethylene, trifluoroethylene, hexafluoroisobutylene, perfluoroalkyl ethylene, fluorovinyl ether, vinyl fluoride, vinylidene fluoride, perfluoro-2,2-dimethyl-1,3-dioxole, perfluoro-2-methylene-4-methyl-1,3-dioxolane, perfluoro(allyl vinyl ether), perfluoro(butenyl vinyl ether), and 2-(allyl)hexafluoroisopropanol.

11. The method of claim 8, wherein the fluorinated monomer comprises at least one monomer selected from the group consisting of: 2,3,4,5,6-pentafluorostyrene, 2-fluorostyrene, 3-fluorostyrene, 4-fluorostyrene, 2,6-difluorostyrene, 2-(trifluoromethyl)styrene, 3-(trifluoromethyl)styrene, and 4-(trifluoromethyl)styrene, 2,2,2-trifluoroethyl methacrylate, 1H, 1H-perfluoro-n-octyl acrylate, 1H,1H-perfluoro-n-decyl acrylate, 1H,1H-perfluoro-n-octyl methacrylate, 1H,1H-perfluoro-n-decyl methacrylate, hexafluoroisopropyl acrylate, and hexafluoroisopropyl methacrylate.

12. The method of claim 8, wherein the hydrophilic monomer comprises at least one monomer selected from the group consisting of: N-vinylformamide, acrylates, and polyethylene glycol-acrylates.

13. A nanoparticle composition comprising:
a fluorinated monomer component; and
a hydrophilic monomer component.

14. The nanoparticle composition of claim 13, wherein the fluorinated monomer component and the hydrophilic monomer component are associated through a crosslinker.

15. The nanoparticle composition of claim 13, wherein the fluorinated monomer component comprises or is dervied from at least one fluorinated monomer selected from the group consisting of: tetrafluoroethylene, hexafluoropropylene, chlorotrifluoroethylene, trifluoroethylene, hexafluoroisobutylene, perfluoroalkyl ethylene, fluorovinyl ether, vinyl fluoride, vinylidene fluoride, perfluoro-2,2-dimethyl-1,3-dioxole, perfluoro-2-methylene-4-methyl-1,3-dioxolane, perfluoro(allyl vinyl ether), perfluoro(butenyl vinyl ether), and 2-(allyl)hexafluoroisopropanol.

16. The nanoparticle composition of claim 13, wherein the fluorinated monomer component comprises or is dervied from at least one fluorinated monomer selected from the group consisting of: 2,3,4,5,6-pentafluorostyrene, 2-fluorostyrene, 3-fluorostyrene, 4-fluorostyrene, 2,6-difluorostyrene, 2-(trifluoromethyl)styrene, 3-(trifluoromethyl)styrene, and 4-(trifluoromethyl)styrene, 2,2,2-trifluoroethyl methacrylate, 1H,1H-perfluoro-n-octyl acrylate, 1H,1H-perfluoro-n-decyl acrylate, 1H,1H-perfluoro-n-octyl methacrylate, 1H,1H-perfluoro-n-decyl methacrylate, hexafluoroisopropyl acrylate, and hexafluoroisopropyl methacrylate.

17. The nanoparticle composition of claim 13, wherein the hydrophilic monomer component comprises or is dervied from at least one hydrophilic monomer selected from the group consisting of: N-vinylformamide, acrylates, and polyethylene glycol-acrylates.

18. The nanoparticle composition of claim 13, further comprising a targeting moiety.

19. The nanoparticle composition of claim 13, wherein the targeting moiety comprises an antibody or RNA fragment.

20. The nanoparticle composition of claim 13, wherein the particle size is about 1000 nanometers or less.

21. The nanoparticle composition of claim 13, wherein the particle size is about 500 nanometers or less.

22. The nanoparticle composition of claim 13, further comprising a fluorescent compound.
